(19) United States
(12) Patent Application Publication
(10) Pub. No.: US 2021/0244830 A1
(43) Pub. Date: Aug. 12, 2021

(54) TMEM-MCD IN THE MINIMALLY INVASIVE ASSESSMENT OF THE ACTIVITY STATUS OF TMEM IN ITS DISSEMINATION OF TUMOR CELLS

(71) Applicants: Albert Einstein College of Medicine, Bronx, NY (US); MONTEFIORE MEDICAL CENTER, Bronx, NY (US)

(72) Inventors: John CONDEELIS, Bronx, NY (US); Craig BRANCH, Bronx, NY (US); Maja OKTAY, Bronx, NY (US)

(21) Appl. No.: 16/972,798

(22) PCT Filed: Jun. 12, 2019
(86) PCT No.: PCT/US19/36847

§ 371 Date: Dec. 7, 2020

Related U.S. Application Data
(60) Provisional application No. 62/683,692, filed on Jun. 12, 2018.

(51) Int. Cl. A61K 49/10

(52) U.S. Cl. CPC A61K 49/105

(57) ABSTRACT

Methods are provided for measuring the activity of TMEM sites in a tumor comprising measuring a transient increase in permeability of blood vessels at TMEM sites that allows tumor cells to enter the blood vessels, wherein permeability is measured using a modality selected from the group consisting of MRI, PET, CT, and SPECT, and wherein a transient increase in permeability indicates that a TMEM site is active. The method can include, for example, obtaining a MenaINV score assessed by fine needle aspiration in the same tissue. The present invention can be used as both a prognostic for dissemination and a predictive end point for identification and validation of dissemination inhibitors/anti-metastasis drugs.

### PRIORITY CLAIM

The present application claims priority to U.S. Provisional Application No. 62/683,692, filed Jun. 12, 2018.

### STATEMENT OF GOVERNMENT SUPPORT

This invention was made with government support under grant number CA100324 awarded by the National Institutes of Health. The government has certain rights in the invention.

### BACKGROUND OF THE INVENTION

Throughout this application various publications are referred to in parentheses. Full citations for these references may be found at the end of the specification. The disclosures of all publications, patents and patent applications mentioned herein are hereby incorporated by reference in their entirety into the subject application to more fully describe the art to which the subject invention pertains.

Metastasis-initiating tumor cells can disseminate from the primary tumor and from secondary tumors in lymph nodes and other sites (4) to spread the tumor systemically, which leads to death of the patient. The cells disseminate through a doorway named the Tumor Micro-Environment for Metastasis (TMEM) doorway (14, 15). The doorway has been investigated using multiphoton imaging of living mice with breast tumors (3) and clinically validated (1, 2).

TMEM assemble with the help of the immune system using macrophages (white cells) to build the doorway and suppress immune rejection of the tumor cells. The TMEM doorway contains a stable group of three cells in direct contact: a tumor cell that expresses a high level of Mena (a signaling molecule essential for tumor cell motility), a proangiogenic Tie2+ macrophage and a vascular endothelial cell (3, 14). TMEM is the only site where metastasis-initiating tumor cells enter blood vessels. When the TMEM doorway opens to allow tumor cells to enter blood vessels, there is a transient increase in permeability of the blood vessel at TMEM that can be seen by optical imaging as the local release of serum components into the tissue. The transient release of serum upon TMEM opening is called the “burst” (3).

The TMEM structure itself, as well as the gene expression pattern of tumor cells at TMEM called MenaCalc (MenaINV-Hi and Mena 11a-Lo), have been validated as prognostic markers for predicting metastasis in breast cancer patients (1, 2, 5-7, 14-15). Given the central importance of TMEM in disseminating tumor cells via blood vessels, a TMEM inhibitor (rebastinib) was developed that prevents dissemination of tumor cells in both mouse models of breast cancer (8, 9) and breast cancer patients (clinical trial led by Drs. Anampa and Sparano NCT02824575).

While studying TMEM function longitudinally during breast cancer treatment, in the residual breast cancers of patients treated with neoadjuvant chemotherapy (NAC) (the standard treatment of paclitaxel following doxorubicin plus cyclophosphamide), it was found that TMEM score, and its associated MenaCalc expression pattern were significantly increased. This was a surprise because previous studies of cohorts of mostly Caucasian patients (>85%) did not detect changes in distant recurrence in response to NAC (10). However, in the Montefiore patient cohort (mostly Latino and African American), NAC, compared to adjuvant chemotherapy, was associated with worse distant recurrence-free survival (22).

In mice with breast cancer, chemotherapy-induced TMEM activity (measured as multiphoton imaging of the burst), and cancer cell dissemination (measured as circulating tumor cells in the blood, aka CTCs) were inhibited by either oral administration of the TMEM inhibitor rebastinib or knockdown of the Mena gene (11). A clinical trial based on this finding was designed using oral rebastinib to inhibit TMEM function (CTCs) and the results were dramatic; most patients achieved complete inhibition of tumor cell dissemination by rebastinib during chemotherapy (clinical trial led by Drs. Anampa and Sparano NCT02824575).

Three of the most important overarching problems in solid tumor (e.g., breast, lung, prostate, pancreatic) management include: (i) preventing dissemination of tumor cells from the primary tumor to distant metastatic sites, (ii) the dearth of therapeutic approaches to prevent or treat metastasis, and (iii) the limited availability of predictive and pharmaco-dynamic biomarkers to assess anti-metastatic drug performance.

Current end points for assessment of treatment response of solid malignancies listed above are inhibition of growth and/or tumor shrinkage as described under Response Evaluation Criteria in Solid Tumors (RECIST) criteria. These end points do not address tumor cell dissemination leading to metastasis that can occur from both primary and secondary tumors. It is becoming increasingly clear that mechanisms behind tumor growth and tumor dissemination are not directly linked during progression and that additional markers, which are prognostic of dissemination and predictive of treatment response to dissemination inhibitors, are needed in the clinical treatment of metastatic disease. The present invention addresses this need.

### SUMMARY OF THE INVENTION

The present invention provides methods of measuring the activity of TMEM sites in a tumor comprising measuring a transient increase in permeability of blood vessels at TMEM sites that allows tumor cells to enter the blood vessels, wherein permeability is measured using a modality selected from the group consisting of MRI, PET, CT, and SPECT, and wherein a transient increase in permeability indicates that a TMEM site is active.

The method can further comprise, for example, obtaining a MenaINV score assessed by fine needle aspiration in the same tissue.

The present invention can be used as both a prognostic for dissemination and a predictive end point for identification and validation of dissemination inhibitors/anti-metastasis drugs.

### BRIEF DESCRIPTION OF THE DRAWINGS

FIGS. 1A-1E depict a graphical overview of a calculation procedure to measure permeability. The panels at left in FIG. 1A depict gradient echo (GE) images collected at varying Flip Angles (FA) to give a curve, similar to the example curve depicted in the graph at right, at every voxel in the image. FIG. 1B depicts a processed T1 map generated using Equation 1 (below), based on the Ernst formula, to fit the estimation of longitudinal relaxation (T1) at every voxel resulting from FIG. 1A, providing a T1 map. The baseline T1 is used to make a two-point estimate of T1 during a dynamic GE acquisition. FIG. 1C depicts dynamic GEs collected to visualize the uptake and clearance of the MRI contrast agent before and following a rapid bolus infusion. FIG. 1D depicts a graph (left) showing the T1 baseline and dynamic contrast changes observed in the GE images (right) used with Equation 2 (below) to estimate the concentration of Mill contrast agent (shown for Gd) over time. A signal representing the arterial concentration of the contrast agent is found in the Gd Concentration image (left). The arterial function represents the input concentration function of contrast agent in the arteries and to the tissue and is used to deconvolve the tissue leakage of contrast agent into the subject's tissues. The right panel of FIG. 1E depicts an example of a tumor voxel in the Gd Concentration image as it varies over time. The left panel of FIG. 1E depicts all voxels in the Gd Concentration image deconvolved, following equation 3 (below), to estimate the permeability map.

$$s = {m}_{0} ( sin FA ) \frac{1 - {e}^{- TR / T {1}_{0}}}{1 - ( cos FA ) * {e}^{- TR / T {1}_{0}}} , \qquad Equation 1$$

where s=signal intensity, TR=repetition time, T1₀=Baseline T1, FA=flip angle, and m₀—longitudinal magnetization.

$$C ( t ) = \frac{\frac{1}{T 1 ( t )} - \frac{1}{T {1}_{0}}}{R 1} , \frac{1}{T 1 ( t )} = \frac{- 1}{TR} * ln ( \frac{1 - D}{1 - ( cos FA ) * D} ) , where D = \frac{S ( t ) - S ( 0 )}{{m}_{0} sin FA} + \frac{1 - {e}^{- TR / T {1}_{0}}}{1 - ( cos FA ) * {e}^{- TR / T {1}_{0}}} , \qquad Equation 2$$

C₍ₜ₎=concentration at time t, S₍ₜ₎=intensity signal at time t, and R1=the relaxivity of the Contrast Agent.

Cₜ(t)=k_{f}_{ₚ}∫₀^{t}Cₐ(t′)dt′, Equation 3

where Cₐ=concentration of Gd in the artery, Cₜ=concentration of Gd in the tissue, k_{f}_{ₚ}=permeability of contrast agent, measured here during the first passage of arterial contrast agent through the tissue (i.e., the “first pass”).

FIGS. 2A-2L. FIG. 2A is a schematic illustration and cohort composition of the MMTV-PyMT Early vs Late stage (EC vs LC) spontaneously metastasizing mammary carcinoma. FIG. 2B depicts histology of EC and LC in MMTV-PyMT mice, used in the study. FIG. 2C depicts representative MRI permeability maps of 1 EC and 1 LC case. FIG. 2D depicts a frequency histogram of the permeability between the EC and LC cases. The threshold is a representation of the threshold in the MRI features defined in FIG. 2E, where values above the threshold are potential TMEM and values below are background. FIG. 2E depicts equations defining TMEM activity as MRI features (Perm^{MEAN} and Uₜₕ). FIGS. 2F and 2G depict charts displaying TMEM activity where FIG. 2F depicts the mean permeability (Perm^{MEAN}) of EC and LC. FIG. 2G is a chart depicting the upper threshold ratio (Uₜₕ) of EC and LC. FIG. 2H depicts representative TMEM stained sections from the EC and LC cases. Circles indicate TMEM. FIG. 2I is a chart depicting the quantification of TMEM score from stained sections in EC and LC cases. FIGS. 2J and 2K are charts depicting the Pearson correlation coefficient between TMEM score from stained sections and Perm^{MEAN} (FIG. 2J) or Uₜₕ (FIG. 2K) among EC and LC cases showing the relationships between TMEM structures and activities PernMean and Uₜₕ, and (FIG. 2L) between Uₜₕ and TMEM activity defined as permeability of intravenous dextran at TMEM structures scored as extravascular dextran.

FIGS. 3A-3E. Evaluation of TMEM-MRI assay sensitivity and its values as a companion diagnostic is illustrated by using TMEM-MRI to detect the effect of the commercially available TMEM inhibitor Rebastinib in PyMT mouse model. FIG. 3A depicts a schematic illustration and cohort composition of the MMTV-PyMT Control vs Rebastinib (Ctrl-Reb) transplantation model. FIG. 3B is a chart depicting upper threshold ratio (Uₜₕ) of Ctrl- and rebastinib (Reb)-treated tumors. FIG. 3C depicts extravascular dextran assay, showing TMEM association with leaky (lower row), but no association of TMEM with non-leaky (upper row) blood vessel. FIG. 3D is a chart depicting the comparison between extravascular dextran leakiness between Ctrl and Reb-treated tumors. FIG. 3E is a chart depicting circulating tumor cells in mice treated with rebastinib or vehicle control. Note that Uₜₕ in 3B predicts results in 3D and E.

FIGS. 4A-4C. Correlation of Mena isoform expression with TMEM score. FIG. 4A depicts TMEM, the intravasation doorway of metastasis-initiating cancer cells as visualized by immunohistochemistry from patient ductal carcinomas (IDC) of the breast. T, Mena-expressing tumor cells; E, CD31-expressing vascular endothelial cells; M, CD68-expressing macrophages (scale bar=300 μm). FIGS. 4B and 4C depicts scatter plots of relative MenaINV transcript expression in FNAs against TMEM score as measured by qRT-PCR and immunohistology, respectively as in FIG. 4A in the entire cohort of 100 IDCs patients (FIG. 4B) or by clinical subtype (FIG. 4C). Data were analyzed by rank-order correlation (n=number of tumor cases). Note positive correlation of MenaINV with TMEM in all subtypes compared to negative correlation with the metastasis suppressor Mena 11a.

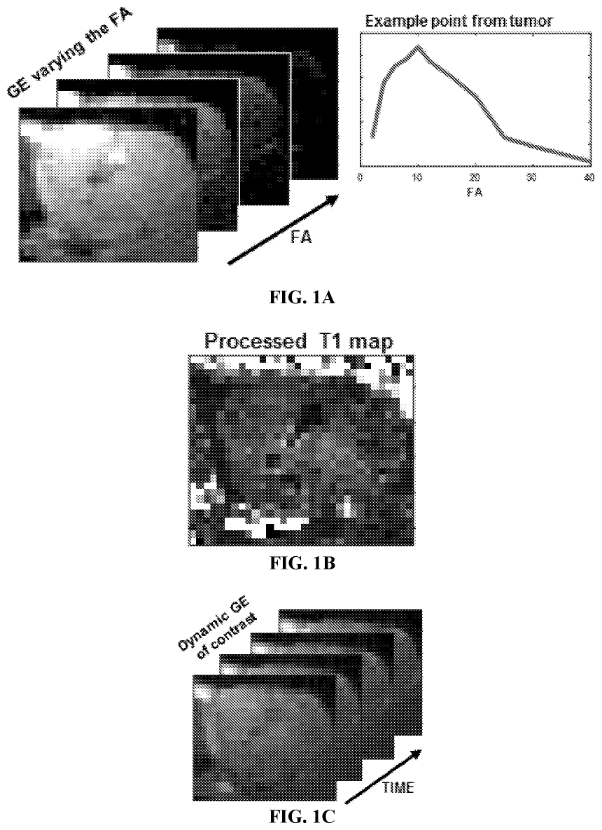

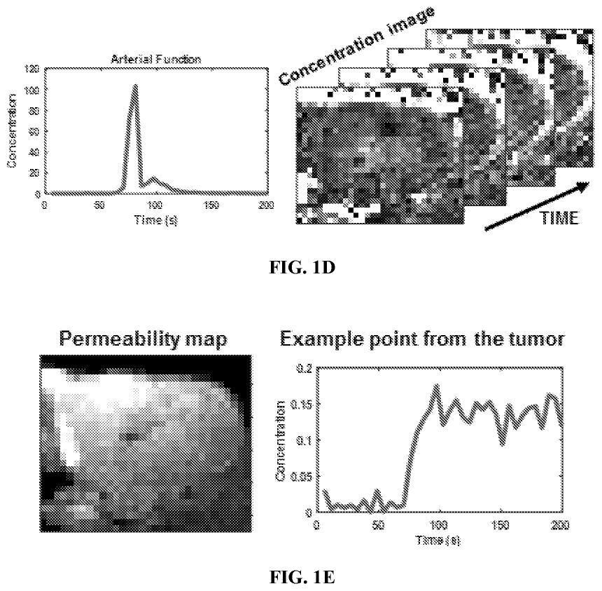

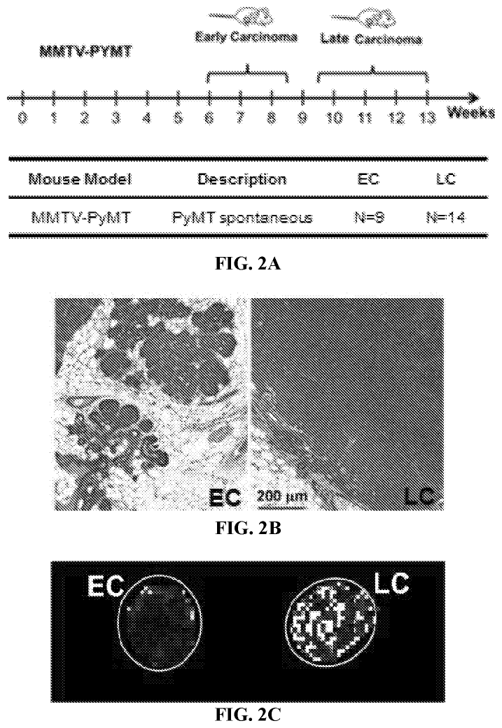

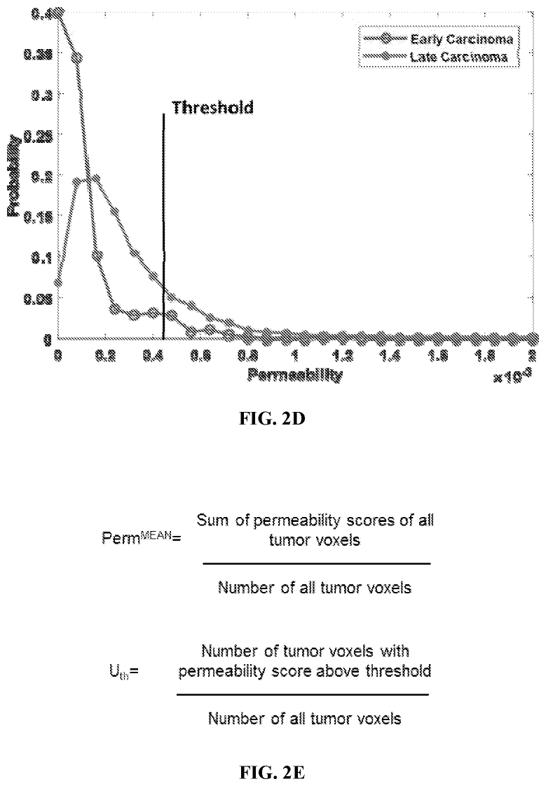

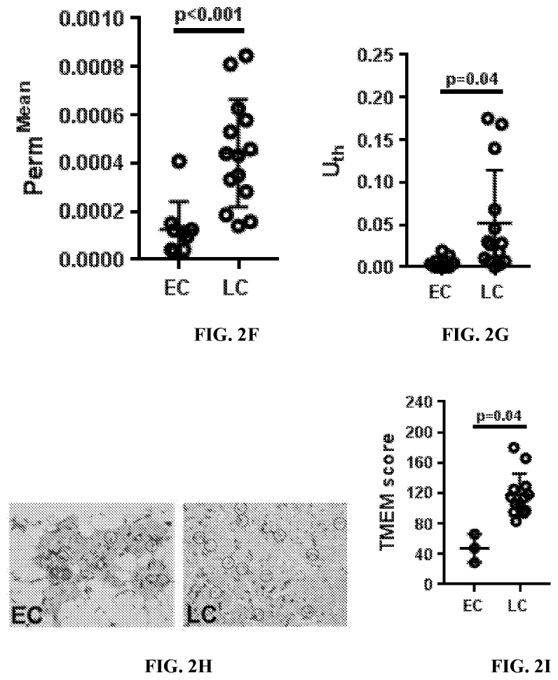

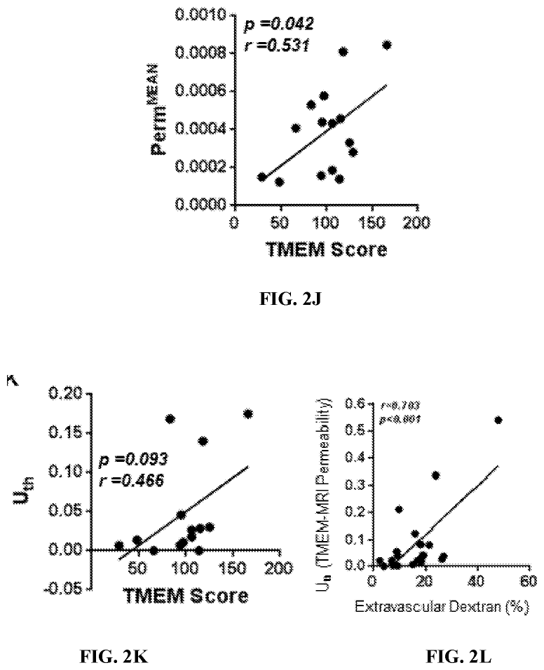

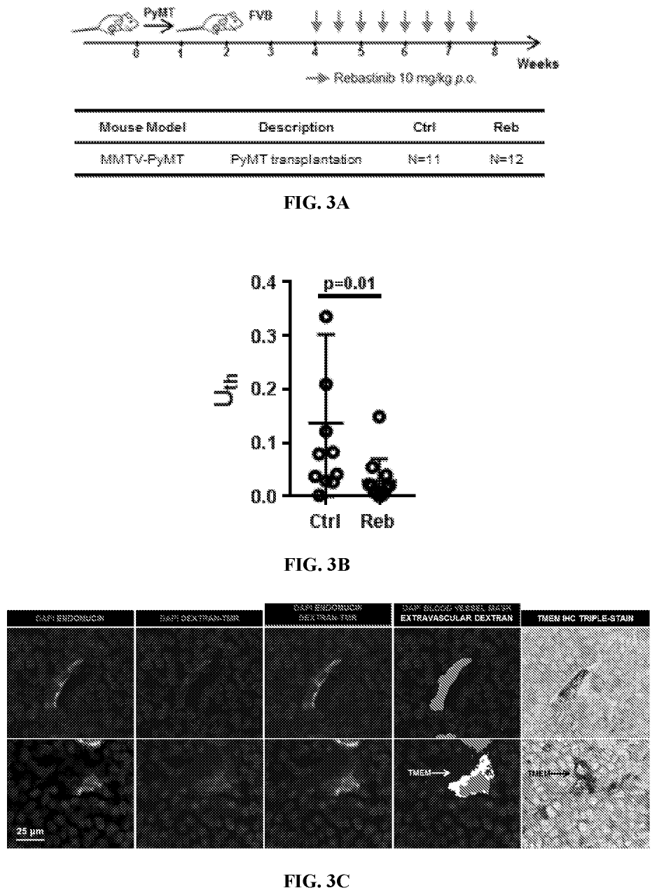

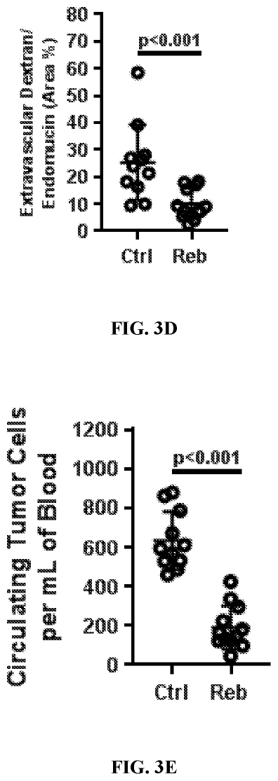

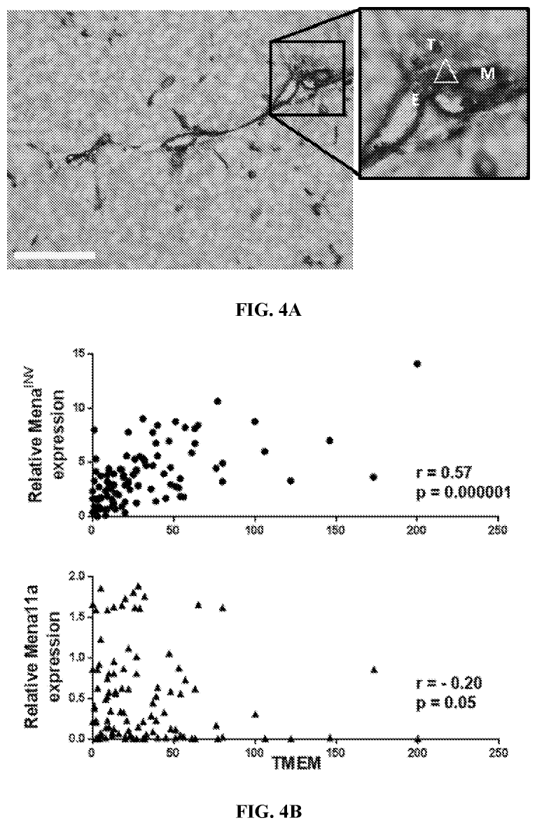

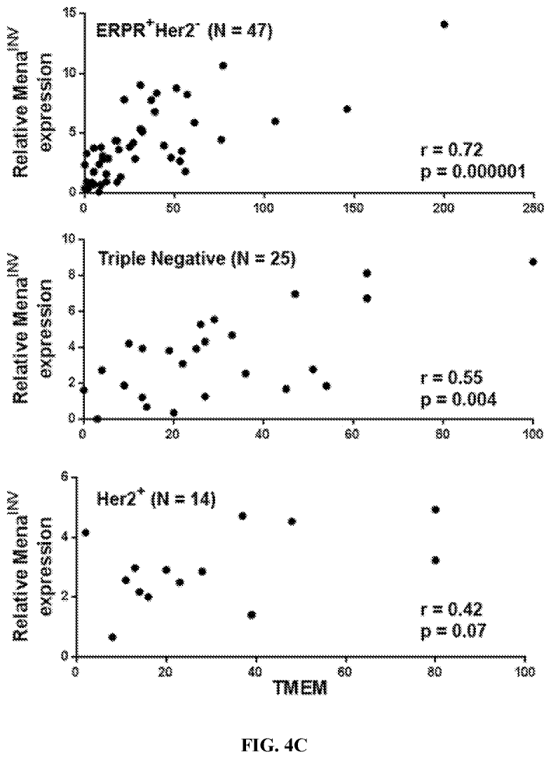

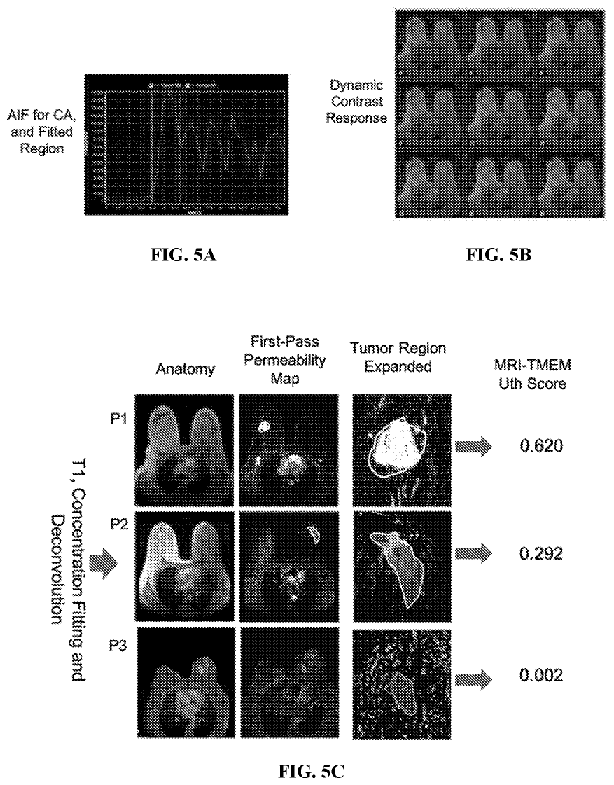

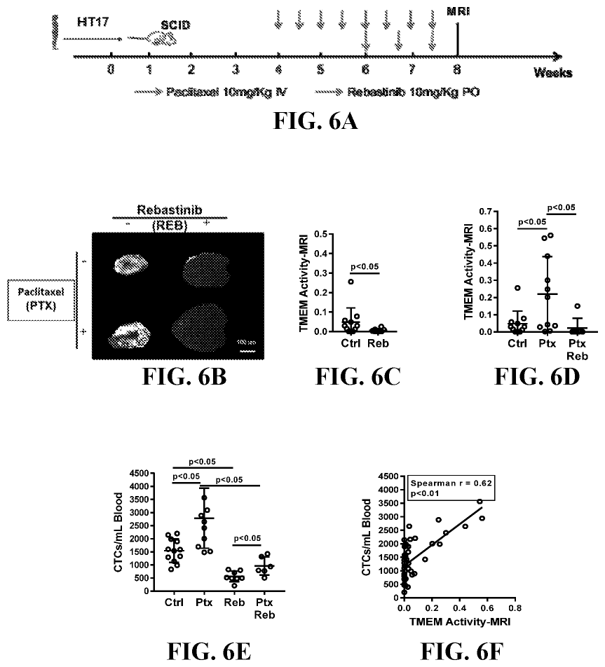

FIGS. 5A-5C. TMEM-MRI assay as practiced in breast cancer patients. MRI assessment similar to the methods detailed above in FIG. 1, with two exceptions. Data acquisition in patients uses a 4D rapid T1-w acquisition with between 2.3 and 4.3 second temporal resolution, T1 calculation uses 4 points with the last point being the dynamic image (with largest flip angle) and equation 4 (deconvolution) also include a term to account for vascular compartment signal contributions, yielding vascular volume; Cₜ(t)=Vₑ*Cₐ(t)+k_{fp}∫₀^{t}Cₐ(t′)dt′. This change to the equation is required to compensate for vascular signal which is T1 visible at field strengths used for human MRI. At higher fields, T2 contrast dominates the arterial signal during the first-pass and does not contribute substantially to the tissue signal. FIG. 5 illustrates the arterial signal (FIG. 5A) obtained from an arterial source (in this instance the heart), the dynamic tissue contrast evolution signal (FIG. 5B) obtained over both breasts with high temporal and spatial resolution, and (FIG. 5C) examples of the mathematical analyses yielding the first-pass permeability (middle column), the regions used for Uₜₕ calculation (third column) and the Uₜₕ measurement. The detailed legend for FIG is as follows: Illustrating for 3 Breast-Tumor Patients the implementation of the TMEM-MRI data acquisition and analyses. FIG. 5A is a graph depicting Arterial Input Function (AIF) of Contrast Agent (CA) obtained from the Left Ventricle of the Heart. Vertical red lines indicate region used for ‘Frist Pass’ permeability assessment. FIG. 5B depicts ynamic Contrast Images obtained beginning before and for 2 minutes following CA infusion. Every third image is displayed (time evolving to right and down) with each image obtained in 4.3 seconds. Note preferential uptake by tumor. FIG. 5C depicts slice of anatomy through tumor (Column 1), first-pass permeability assessment (column 2) and expanded view of region used for TMEM-MRI Uₜₕ (Column 3). ROI of tumor determined from anatomical T2W pre-contrast image. Uₜₕ score as defined in FIG. 2D, using 0.8E-03 as threshold.

FIGS. 6A-6F. Chemotherapy-induced increase of TMEM-dependent vascular permeability can be captured by MRI in primary breast tumors of patients. FIG. 6A is a schematic illustration and experimental drug treatment. FIG. 6B depicts representative permeability maps of a control, chemo (PTX) and a rebastinib-treated case in chemo and no-chemo settings. FIG. 6C is a chart depicting quantification of the TMEM Activity-MRI (Uₜₕ) of control and rebastinib groups. FIG. 6D depicts a chart-based assessment of TMEM Activity-MRI (Uₜₕ) in response to chemotherapy-induced prometastatic effects and response to rebastinib in the primary tumor. FIG. 6E depicts a chart comparing circulating tumor cells when treated with rebastinib or vehicle control. FIG. 6F depicts a chart displaying the correlation between circulating tumor cells and TMEM Activity-MRI in controls. Note that rebastinib is used here to illustrate the use of TMEM-MRI as a companion diagnostic for any of the agents mentioned in the claim, and that rebastinib is not part of the invention.

### EXPERIMENTAL DETAILS

The invention provides a method for measuring the activity of Tumor Micro-Environment for Metastasis (TMEM) sites in a tumor comprising:

measuring a transient increase in permeability of blood vessels at TMEM sites that allows tumor cells to enter the blood vessels, wherein permeability is measured using a modality selected from the group consisting of magnetic resonance imaging (MRI), positron emission tomography (PET), computed tomography (CT), and single-photon emission computerized tomography (SPECT), and wherein a transient increase in permeability indicates that a TMEM site is active.

Vasculature permeability can be detected, e.g., by local release of serum components into surrounding tissue. For example, permeability can be measured using MRI contrast agents and/or magnetic particles, such as, e.g., a gadolinium-based MRI contrast agent.

The method can further comprise measuring expression of one or more of MenaINV, pan-Mena, Mena11a, CD31 and CD68 in cells of the tumor that is imaged. An endothelial cell of the TMEM can be detected by detecting CD31. A macrophage of the TMEM can be detected by detecting CD68. An invasive tumor cell of the TMEM can be detected by measuring MenaINV or pan-Mena minus Mena11a (MenaCalc). A sample of cells can be obtained from the tumor using, e.g., fine needle aspiration (FNA). FNA is a method of tissue collection yielding a >95% pure population of tumor cells. It is important because it provides a minimally invasive method which can be used before more extensive tissue collection by core biopsy, as well as during treatment, or from metastatic sites which are usually not surgically samples of treated.

In a preferred embodiment, TMEM activity is detected using magnetic resonance and/or magnetic particle-based contrast detection combined with MenaINV score assessed by fine needle aspiration in the same tissue. The term TMEM-MCD is used for the detection of TMEM activity using minimally invasive contrast-based detection using magnetic resonance and/or magnetic particle-based contrast detection combined with MenaINV score assessed by fine needle aspiration in the same tissue.

TMEM activity can be expressed as one or more of a Perm^{MEAN} score and a Uₜₕ score, wherein

Perm^{MEAN} represents the sum of permeability scores of all tumor voxels divided by the number of all tumor voxels, and

Uₜₕ represents the number of tumor voxels with permeability scores above threshold divided by the number of all tumor voxels.

TMEM activity can be further expressed as a Perm^{MEAN} score or a Uₜₕ score relative to one or more of a TMEM score obtained by immunohistochemistry, a MenaINV score and a MenaCalc score.

A TMEM can be defined, for example, by juxtaposition of a macrophage, an endothelial cell and an invasive tumor cell, wherein an invasive tumor cell is identified by expression of high pan-Mena, MenaCalc and/or MenaINV (see, e.g., 14).

A TMEM can also be defined, for example, by a Tie2Hi/VEGFHi (e.g., VEGFAHi) macrophage in direct contact with a blood vessel with decreased VE-Cadherin and/or ZO-1 endothelial staining (see, e.g., 15).

TMEM assembly and function is mechanistically linked to the expression pattern of Mena isoforms where total Mena expression minus the expression of the metastasis suppressor Mena11a (MenaCalc=pan (all) Mena−Mena11a) is associated with TMEM assembly. MenaCalc is independently predictive of metastatic recurrence and survival in breast cancer patients and is predictive of response to standard forms of chemotherapy (5-7).

In addition, the MenaINV isoform is associated with increased receptor tyrosine kinase (RTK) sensitivity and invadopodium assembly, two events linked to efficient TMEM function. MenaINV levels are associated with increased TMEM function and metastatic risk and are predictive of the response to standard forms of chemotherapy (6, 16-19).

Multiplex staining can be used to stain TMEM, and MenaINV or pan-Mena & Mena11a. Endothelial cells can be detected, for example, using an agent that is specific for CD31. Macrophages can be detected, for example, using an agent specific for CD68, Tie2 and/or CD206. Invasive tumor cells can be detected, for example, using an agent specific for panMena or MenaINV.

The endothelial cells, macrophages, and/or invasive tumor cells can be detected using antibodies, monoclonal antibodies, antibody fragments, peptides, aptamers and/or cDNA probes that are specific for their target.

As used herein, the term “antibody” encompasses whole antibodies and fragments of whole antibodies wherein the fragments specifically bind to endothelial cells, macrophages, panMena, MenaINV or Mena11a. Antibody fragments include, but are not limited to, F(ab′)2 and Fab′ fragments and single chain antibodies. F(ab′)2 is an antigen binding fragment of an antibody molecule with deleted crystallizable fragment (Fc) region and preserved binding region. Fab′ is ½ of the F(ab′)2 molecule possessing only ½ of the binding region. The term antibody is further meant to encompass polyclonal antibodies and monoclonal antibodies. Antibodies may be produced by techniques well known to those skilled in the art. Polyclonal antibody, for example, may be produced by immunizing a mouse, rabbit, or rat with purified polypeptides encoded by the variants of Mena. Monoclonal antibody may then be produced by removing the spleen from the immunized mouse and fusing the spleen cells with myeloma cells to form a hybridoma which, when grown in culture, will produce a monoclonal antibody. The antibody can be, e.g., any of an IgA, IgD, IgE, IgG, or IgM antibody. The IgA antibody can be, e.g., an IgA1 or an IgA2 antibody. The IgG antibody can be, e.g., an IgG1, IgG2, IgG2a, IgG2b, IgG3 or IgG4 antibody. A combination of any of these antibodies' subtypes can also be used. One consideration in selecting the type of antibody to be used is the size of the antibody. For example, the size of IgG is smaller than that of IgM allowing for greater penetration of IgG into tissues. The antibody can be a human antibody or a non-human antibody such as a goat antibody or a mouse antibody. Antibodies can be “humanized” using standard recombinant DNA techniques.

Human MenaINV and Mena 11a sequences are indicated below:

MenaINV
(SEQ ID NO: 1)
AQSKVTATQD STNLRCIFC,

gcccagagca aggttactgc tacccaggac agcactaatt tgcgatgtat tttctgt (SEQ ID NO:2);

Mena11a
(SEQ ID NO: 3)
RDSPRKNQIV FDNRSYDSLH R,

acgggattct ccaaggaaaa atcagattgt ttttgacaac aggtcctatg attcattaca cag (SEQ ID NO:4).

Aptamers are single stranded oligonucleotides or oligonucleotide analogs that bind to a particular target molecule, such as a protein. Thus, aptamers are the oligonucleotide analogy to antibodies. However, aptamers are smaller than antibodies. Their binding is highly dependent on the secondary structure formed by the aptamer oligonucleotide. Both RNA and single stranded DNA (or analog) aptamers can be used. Aptamers that bind to virtually any particular target can be selected using an iterative process called SELEX, which stands for Systematic Evolution of Ligands by EXponential enrichment.

The agent that specifically binds to macrophages, endothelial cells, panMena, MenaINV or Mena11a can be labeled with a detectable marker. Labeling may be accomplished using one of a variety of labeling techniques, including peroxidase, chemiluminescent, fluorescence and/or radioactive labels known in the art. The detectable marker may be, for example, a nonradioactive or fluorescent marker, such as biotin, fluorescein (FITC), acridine, cholesterol, or carboxy X rhodamine, which can be detected using fluorescence and other imaging techniques readily known in the art. Alternatively, the detectable marker may be a radioactive marker, including, for example, a radioisotope. The radioisotope may be any isotope that emits detectable radiation, such as, for example, ³⁵S, ³²P, or ³H. Radioactivity emitted by the radioisotope can be detected by techniques well known in the art. For example, gamma emission from the radioisotope may be detected using gamma imaging techniques, particularly scintigraphic imaging.

The expression of Mena can be normalized relative to the expression of protein variants that are not changed in expression in a metastatic tumor. Examples of proteins that could be used as controls include those of the Ena/VASP family that are unchanged in their expression in metastatic cells. Other examples of proteins or genes that could be used as controls include those listed as relatively unchanged in expression in disseminating tumor cells (20, 21). Such controls include N-WASP, Rac1, Pak1, and PKCalpha and beta.

The tumor can be any tumor, for example, a breast, pancreas, prostate, colon, brain, liver, lung, head or neck tumor. The tumor can be, for example, a secretory epithelial tumor, a mesenchymal derived tumor such as Ewing's sarcoma, or another neuroendocrine tumor such as a pancreatic neuroendocrine neoplasm or any small blue cell tumor.

The invention also provides a method of assessing effectiveness of a treatment for metastatic cancer in a subject comprising:

a) obtaining a first TMEM activity score by any of the methods disclosed herein before treatment of the subject or at a first stage of treatment of the subject;

b) obtaining a second TMEM activity score after treatment of the subject or at a second stage of treatment of the subject; and

c) comparing the scores obtained in step a) and step b),

wherein a decrease in the TMEM activity score after treatment of the subject indicates that the treatment is effective in treating metastatic cancer or in decreasing the likelihood of a cancer to metastasize; and

wherein an increase in the TMEM activity score indicates a need to continue treatment and/or switch to a different treatment.

The treatment can be, for example, a cytotoxic chemotherapy drug, a receptor tyrosine kinase (RTK) inhibitor, a (TK) tyrosine kinase inhibitor, or combinations thereof. The RTK inhibitor can be, for example, an EGFR, HGFR, IGFR, CSF1R, Tie2 or VEGFR inhibitor, or combinations thereof. The TK inhibitor can be, for example, a Src, Abl or Arg inhibitor, or combinations thereof. The treatment can comprise, for example, administration of rebastinib (4-[4-[(5-tert-butyl-2-quinolin-6-ylpyrazol-3-yl)carbamoylamino]-3-fluorophenoxy]-N-methylpyridine-2-carboxamide), an anti-tubulin chemotherapy, a taxane (e.g. paclitaxel), a non-taxane microtubule inhibitors (e.g. eribulin), a topoisomerase inhibitor (e.g. etoposide), an intercalating agent (e.g. doxorubicin), a DNA cross-linking agent (e.g. cisplatin), an alkylating agent (e.g. cyclophosphamide), a vascular endothelial growth factor (VEGF) inhibitor, antibody or blocking antibody, a colony stimulating factor 1 (CSF1) receptor inhibitor, or combinations thereof. The treatment can be, or comprise, radiation.

The invention also provides a method for assessing the prognosis of a subject undergoing treatment for a tumor, the method comprising obtaining a TMEM activity score by any of the methods disclosed herein at different time points during treatment, wherein an increase in the score over time indicates a worsening of the subject's prognosis.

A method for determining a course of treatment for a tumor for a subject, the method comprising obtaining a TMEM activity score by any of the methods disclosed herein, wherein a high TMEM activity score indicates that the subject is at increased risk of hematogenous metastasis and should be treated for a metastatic tumor.

The invention also provides method of treating a subject for a hematogenous metastatic cancer comprising:

a) receiving an indication that the subject has a hematogenous metastatic cancer or a likelihood of tumor cells undergoing hematogenous metastasis, wherein the subject was diagnosed by any of the methods disclosed herein; and

b) administering an anti-metastatic therapy to the subject identified as having a hematogenous metastatic cancer or a likelihood of tumor cells undergoing hematogenous metastasis.

The invention further provides a method of treating a patient comprising:

a) ordering a diagnostic test performed by any of the methods disclosed herein, and

b) treating the patient based on the results of the diagnostic test;

wherein a test result indicating that the patient has a hematogenous metastatic cancer or that tumor cells of the patient are likely undergoing hematogenous metastasis requires aggressive anti-cancer therapy.

The treatment or therapy can comprise, for example, one or more of a cytotoxic chemotherapy drug, a receptor tyrosine kinase (RTK) inhibitor, a (TK) tyrosine kinase inhibitor, an EGFR, HGFR, IGFR, CSF1R, Tie2 or VEGFR inhibitor, a Src, Abl or Arg inhibitor, rebastinib (4-[4-[(5-tert-butyl-2-quinolin-6-ylpyrazol-3-yl)carbamoylamino]-3-fluorophenoxy]-N-methylpyridine-2-carboxamide), an anti-tubulin chemotherapy, a taxane (e.g. paclitaxel), a non-taxane microtubule inhibitors (e.g. eribulin), a topoisomerase inhibitor (e.g. etoposide), an intercalating agent (e.g. doxorubicin), a DNA cross-linking agent (e.g. cisplatin), an alkylating agent (e.g. cyclophosphamide), a VEGF inhibitor, antibody or blocking antibody, a CSF1 receptor inhibitor, radiation and surgery, or combinations thereof.

All combinations of the various elements described herein are within the scope of the invention unless otherwise indicated herein or otherwise clearly contradicted by context.

Where a numerical range is provided herein, it is understood that all numerical subsets of that range, and all the individual integers contained therein, are provided as part of the invention.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

A new assay for TMEM activity has been developed and validated. The TMEM-MRI test measures the activity of TMEM in living subjects with tumors by imaging the blood vascular permeability (burst) associated with opening of the TMEM doorway. Imaging of the burst was conceptualized to use a contrast agent that would exit the vasculature within a TMEM activity-related interval of time through the TMEM facilitated opening of the vascular wall. The inventors recognized that the TMEM facilitated leakage can be visualized by any imaging modality using intravascular contrast agents but chose to prove the invention using MRI. The simplest (albeit perhaps not the most sensitive) contrast agent was chosen to be FDA-approved gadolinium-DTPA (Gd-DTPA) molecules, which provide T1 and T2 contrast at the site of the TMEM. Using intravenously injected Gd-DTPA, in a dynamic and quantitative assessment allowing assessment of the change in tissue permeability induced by TMEM, with adequate temporal and spatial resolution, the permeability change associated with the TMEM occurrence can be measured with high spatial resolution. Magnetic particles could also be used as the contrast agent. Other imaging modalities (PET, CT, and SPECT) with the appropriately chosen contrast agent could be used to visualize TMEM-induced vascular permeability. Other MRI based means affording measurement of the vascular permeability change, including indices of permeability and leaking resulting from the TMEM facilitated burst may also be employed to detect TMEM activity, including the initial tissue transfer rate of contrast from the arterial compartment to the tissue compartment, such as the transfer rate index, or the permeability surface area product. TMEM facilitated increase in tissue diffusion, measured using the apparent diffusion constant or using Diffusion Tensor Imaging (DTI) may also be developed to detect TMEM. Measurement of the efflux of supra-magnetic nanoparticles using Magnetic Particle Imaging (MPI) can also be used to measure TMEM activity. Thus, TMEM-MRI is potentially only one version of the tests that can be used to detect TMEM activity. The TMEM-MRI test was validated successfully in mice (FIGS. 1-4). Using the TMEM-MRI test, it was possible to predict metastatic risk associated with the primary tumor as well as detect the inhibition of TMEM activity by rebastinib (FIGS. 2-3).

The TMEM-MRI test can be used to predict pro-metastatic changes in cancer patients in response to chemotherapy in the neoadjuvant setting (11) and during metastatic disease and should be valuable in treatment decisions at all stages. In addition, the TMEM-MRI test can be used as a companion diagnostic to follow the response in real time to anti-metastasis drugs such as orally administered rebastinib (8).

Measuring the burst associated with TMEM-induced localized vascular permeability using standard MRI contrast agents and/or magnetic particles gives a direct measure of TMEM-associated permeability activity. The simultaneous measure of MenaINV expression in tumor cells obtained by Fine Needle Aspiration (FNA) in the same patient receiving the TMEM-MRI, which gives a measure of TMEM count (FIG. 4) (12) and of the TMEM-associated tumor cell trans-endothelial migration activity (12, 13), constitute a minimally invasive approach to measure tumor cell dissemination at TMEM.

Therefore, the TMEM-MCD invention combines the MRI contrast-based measure of TMEM permeability with FNA-correlated TMEM number and MenaINV expression status in the patient to arrive at a measure of TMEM activity that documents tumor cell dissemination activity associated with TMEM. Thus, TMEM-MRI activity can be expressed as TMEM Uₜₕ/TMEM-MenaINV score derived from MRI and FNA-MenaINV score, respectively.

Currently there are no live or fixed tissue markers for tumor cell dissemination in clinical use. In addition, there are no pharmaco-dynamic biomarkers that can be used as end points for evaluation of dissemination inhibitors. There are prognostic markers that can be used to assess risk of distant recurrence but all of these except one are based on growth markers and not directly related to dissemination. TMEM is the only marker that is used to directly assess the risk of distant recurrence due to dissemination and is based on the number of TMEM anatomical structures present in Formalin-Fixed Paraffin-Embedded (FFPE) primary tumor tissue (1, 2). However, the presence of TMEM does not inform about the activity status of TMEM sites in disseminating tumor cells and its identification is not related to its activity status. The present invention provides information directly about the activity status of TMEM and actively disseminating tumor cells and therefore can be used for the assessment of risk of dissemination of tumor cells, and as an endpoint in the identification and validation of dissemination inhibitors.

240,000 breast cancer patients per year in the USA alone would benefit from application of this test as a primary prognostic and about 30% of these as an endpoint in treatment. The test can be done using standard FDA approved gadolinium-based MRI contrast agents allowing for safe and reliable implementation in radiology clinics.

### REFERENCES

1. Rohan et al. J Natl Cancer Inst 106 (8) (Jun. 3, 2014). PMID: 24895374/PMCID: PMC4133559.

2. Sparano et al. NPJ Breast Cancer 3:42 (Nov. 8, 2017). PMID: 29138761/PMCID: PMC5678158.

3. Harney et al. Cancer Discov 5 (9):932-943 (September 2015). PMID: 26269515/PMCID: PMC4560669.

4. Entenberg et al. Nat Methods 15 (1):73-80 (January 2018). PMID: 29176592/PMCID: PMC5755704.

5. Agarwal et al. Breast Cancer Res 14 (5):R124 (Sep. 12, 2012). PMID: 22971274/PMCID: PMC3962029.

6. Karagiannis et al. J Cell Sci 129 (9):1751-1758 (May 1, 2016). PMID: 27084578/PMCID: PMC4893654.

7. Forse et al. BMC Cancer 15:483 (Jun. 27, 2015). PMID: 26112005/PMCID: PMC4482190.

8. Harney et al. Mol Cancer Ther 16 (11):2486-2501 (November 2017). PMID: 28838996/PMCID: PMC5669998.

9. Karagiannis et al. Oncotarget 8 (67):110733-110734 (Nov. 27, 2017). PMID: 29340008/PMCID: PMC5762276.

10. Rastogi et al. J Clin Oncol 26 (5):778-785 (Feb. 10, 2008). PMID: 18258986.

11. Karagiannis et al. Sci Transl Med 9 (397) (Jul. 5, 2017). PMID: 28679654/PMCID: PMC5592784.

12. Pignatelli et al. Sci Signal 7 (353):ra112 (Nov. 25, 2014). PMID: 25429076/PMCID: PMC4266931.

13. Pignatelli et al. Sci Rep 6:37874 (Nov. 30, 2016). PMID: 27901093/PMCID: PMC5129016.

14. U.S. Pat. No. 8,642,277 B2, issued Feb. 4, 2014 (Condeelis et al.).

15. U.S. Patent Publ. No. US 2018/0106811 A1, published Apr. 19, 2018 (Condeelis et al.).

16. U.S. Pat. No. 8,603,738 B2, issued Dec. 10, 2013 (Condeelis et al.).

17. Eddy et al. Trends Cell Biol 27 (8):595-607 (August 2017). PMID: 28412099/PMCID: PMC5524604.

18. Roussos et al. J Cell Sci 124 (Pt 13):2120-2131 (Jul. 1, 2011). PMID: 21670198/PMCID: PMC3113666.

19. Roussos et al. Clin Exp Metastasis 28 (6):515-527 (August 2011). PMID: 21484349/PMCID: PMC3459587.

20. Condeelis et al. Annu Rev Cell Dev Biol 21:695-718 (2005). PMID: 16212512.

21. Patsialou et al. Breast Cancer Res 14 (5):R139 (Oct. 31, 2012). PMID: 23113900/PMCID: PMC4053118.

22. Pastoriza et al. Clin Exp Metastasis 35 (7):613-623 (October 2018). PMID: 30136072/PMCID: PMC6202136.

1. A method of measuring the activity of TMEM sites in a tumor comprising:
measuring a transient increase in permeability of blood vessels at TMEM sites that allows tumor cells to enter the blood vessels, wherein permeability is measured using a modality selected from the group consisting of MRI, PET, CT, and SPECT, and wherein a transient increase in permeability indicates that a TMEM site is active.

2. The method of claim 1, wherein permeability is detected by local release of serum components into surrounding tissue.

3. The method of claim 1, wherein permeability is measured using MRI contrast agents and/or magnetic particles.

4. The method of claim 1, wherein permeability is measured using a gadolinium-based MRI contrast agent.

5. The method of claim 1, further comprising measuring expression of one or more of MenaINV, pan-Mena, Mena11a, CD31 and CD68 in cells of the tumor that is imaged.

6. The method of claim 5, wherein an endothelial cell of the TMEM is detected by detecting CD31.

7. The method of claim 5, wherein a macrophage of the TMEM is detected by detecting CD68.

8. The method of claim 5, wherein a sample of cells is obtained from the tumor using fine needle aspiration (FNA).

9. The method of claim 5, wherein TMEM activity is detected using magnetic resonance and/or magnetic particle based contrast detection combined with MenaINV score assessed by fine needle aspiration in the same tissue.

10. The method of claim 1, wherein TMEM activity is expressed as one or more of a Perm^{MEAN} score and a Uₜₕ score, wherein
Perm^{MEAN} represents the sum of permeability scores of all tumor voxels divided by the number of all tumor voxels; and
Uₜₕ represents the number of tumor voxels with permeability scores above threshold divided by the number of all tumor voxels.

11. The method of claim 10, wherein TMEM activity is expressed as a Perm^{MEAN} score or a Uₜₕ score relative to one or more of a TMEM score obtained by immunohistochemistry, a MenaINV score and a MenaCalc score.

12. The method of claim 1, wherein the tumor is a breast, pancreas, prostate, colon, brain, liver, lung, head or neck tumor.

13. A method of assessing effectiveness of a treatment for metastatic cancer in a subject comprising:
a) obtaining a first TMEM activity score by the method of claim 1 before treatment of the subject or at a first stage of treatment of the subject;
b) obtaining a second TMEM activity score after treatment of the subject or at a second stage of treatment of the subject; and
c) comparing the scores obtained in step a) and step b),
wherein a decrease in the TMEM activity score after treatment of the subject indicates that the treatment is effective in treating metastatic cancer or in decreasing the likelihood of a cancer to metastasize; and
wherein an increase in the TMEM activity score indicates a need to continue treatment and/or switch to a different treatment.

14. The method of claim 13, wherein the treatment is a cytotoxic chemotherapy drug, a receptor tyrosine kinase (RTK) inhibitor, a (TK) tyrosine kinase inhibitor, or combinations thereof.

15. The method of claim 14, wherein the RTK inhibitor is an EGFR, HGFR, IGFR, CSF1R, Tie2 or VEGFR inhibitor.

16. The method of claim 14, wherein the TK inhibitor is a Src, Abl or Arg inhibitor.

17. The method of claim 14, wherein the treatment comprises administration of rebastinib (4-[4-[(5-tert-butyl-2-quinolin-6-ylpyrazol-3-yl)carbamoylamino]-3-fluorophenoxy]-N-methylpyridine-2-carboxamide), an anti-tubulin chemotherapy, a taxane (e.g. paclitaxel), a non-taxane microtubule inhibitors (e.g. eribulin), a topoisomerase inhibitor (e.g. etoposide), an intercalating agent (e.g. doxorubicin), a DNA cross-linking agent (e.g. cisplatin), an alkylating agent (e.g. cyclophosphamide), a VEGF inhibitor, antibody or blocking antibody, a CSF1 receptor inhibitor, or combinations thereof.

18. The method of claim 14, wherein the treatment is radiation.

19. A method for assessing the prognosis of a subject undergoing treatment for a tumor, the method comprising obtaining a TMEM activity score by the method of claim 1 at different time points during treatment, wherein an increase in the score over time indicates a worsening of the subject's prognosis.

20. A method for determining a course of treatment for a tumor for a subject, the method comprising obtaining a TMEM activity score by the method of claim 1, wherein a high TMEM activity score indicates that the subject is at increased risk of hematogenous metastasis and should be treated for a metastatic tumor.

21. A method of treating a subject for a hematogenous metastatic cancer comprising:
a) receiving an indication that the subject has a hematogenous metastatic cancer or a likelihood of tumor cells undergoing hematogenous metastasis, wherein the subject was diagnosed by the method of claim 1; and
b) administering an anti-metastatic therapy to the subject identified as having a hematogenous metastatic cancer or a likelihood of tumor cells undergoing hematogenous metastasis.

22. A method of treating a patient comprising:
a) ordering a diagnostic test performed by the method of claim 1, and
b) treating the patient based on the results of the diagnostic test;
wherein a test result indicating that the patient has a hematogenous metastatic cancer or that tumor cells of the patient are likely undergoing hematogenous metastasis requires aggressive anti-cancer therapy.

23. The method of claim 20, wherein the treatment or therapy comprises one or more of a cytotoxic chemotherapy drug, a receptor tyrosine kinase (RTK) inhibitor, a (TK) tyrosine kinase inhibitor, an EGFR, HGFR, IGFR, CSF1R, Tie2 or VEGFR inhibitor, a Src, Abl or Arg inhibitor, rebastinib (4-[4-[(5-tert-butyl-2-quinolin-6-ylpyrazol-3-yl)carbamoylamino]-3-fluorophenoxy]-N-methylpyridine-2-carboxamide), an anti-tubulin chemotherapy, a taxane (e.g. paclitaxel), a non-taxane microtubule inhibitors (e.g. eribulin), a topoisomerase inhibitor (e.g. etoposide), an intercalating agent (e.g. doxorubicin), a DNA cross-linking agent (e.g. cisplatin), an alkylating agent (e.g. cyclophosphamide), a VEGF inhibitor, antibody or blocking antibody, a CSF1 receptor inhibitor, radiation and surgery, or combinations thereof.
